(19) United States
(12) Patent Application Publication
(10) Pub. No.: US 2010/0094291 A1
(43) Pub. Date: Apr. 15, 2010

(54) Clamp For A Medical Implant And A Method For Using The Same

(76) Inventor: Damon DelBello, Bedford, NY (US)

Correspondence Address: ST. ONGE STEWARD JOHNSTON & REENS, LLC, 986 BEDFORD STREET, STAMFORD, CT, 06905-5619, US

(21) Appl. No.: 12/248,418

(22) Filed: Oct. 9, 2008

(51) Int. Cl. A61B 17/04; A61B 19/00

(52) U.S. Cl. 606/62; 606/324; 606/301; 128/898

(57) ABSTRACT

A clamping device for use with a medical instrument is disclosed. Specifically, a clamping device for use with an intramedullary rod in children and young adults. The intramedullary rod is inserted into the medullary cavity of a long bone and a portion of the rod protrudes. The disclosed clamping device secures the protruding end of the rod, and reduces movement of the rod in all directions relative to the insertion site of the rod.

### FIELD OF THE INVENTION

The present invention relates to a device for stabilizing a medical implant and a method for using the same. More specifically, the present invention relates to a clamp for securing an end of an intramedullary rod that protrudes from an insertion site in a bone so that movement of the end of the intramedullary rod is reduced.

### BACKGROUND OF THE INVENTION

The use of intramedullary rods (also known as intramedullary nails, and elastic nails) to treat and repair fractures in bones, such as the femur and tibia, is well known. For example U.S. Pat. No. 4,169,470 to Ender et al. discloses an elongated relatively thin, elastic, flexible and resilient rod for emplacement in and along the medullary canal of a fractured bone.

In typical methods of fracture repair, an intramedullary rod is inserted into and along the axis of the medullary cavity of a long bone so that the rod passes across a fracture in the bone. The medullary cavity is the central cavity of the bone shaft (diaphysis). It extends from the proximal end of the bone (located closer to the center of the body) to the distal end of the bone (located further from the center of the body). The medullary cavity is hollow and has walls composed of compact bone. The rod can be inserted into the bone either using an antegrade technique or retrograde technique. Antegrade technique refers to inserting the rod into the proximal end of the bone, in the case of the femur closest to the hip, and retrograde technique refers to inserting the rod into the distal end of the bone, in the case of the femur closest to the knee.

Using an antegrade technique, the intramedullary rod is inserted into the medullary canal through a hole drilled into the proximal end of the bone. Next, the rod is pushed down the axis of the medullary cavity and across the fracture site, and further down the cavity until the leading end of the rod reaches a distal end of the medullary cavity.

Once the rod is fully inserted, it is locked into place at its distal and proximal ends to increase stability, and ensure proper healing of the fracture. At one or both ends of the long bone screws are inserted transversely into the bone such that they are approximately perpendicular to the intramedullary cavity and inserted intramedullary rod. The intramedullary rod includes transverse openings located at is distal and proximal ends for receiving the anchors. The anchors are inserted through the bone, through the transverse openings in either end of the intramedullary rod, and further into the bone. In this way, the anchors lock the intramedullary rod in place in the medullary cavity by fixing one or more of the distal and proximal ends the intramedullary rod. Once installed, this known system locks the intramedullary rod and reduces its movement in the medullary cavity, thus facilitating healing of the fracture.

A disadvantage the intramedullary rod systems described above is that they cannot be used in children and young adults due to the risk of bone and limb deformity resulting from continued growth of the bone while the intramedullary rod remains in the medullary cavity bone. Typically, an intramedullary rod remains in the bone for an indefinite time period, even after a fracture has healed. If an intramedullary rod remains in a bone of a child or young adult, however, bone it could cause deformation of the bone.

In reference to FIG. 1 the epiphysis 10 is shown. The epiphysis 10 in the rounded end of the long bone. The epiphysial plate 12, also known as the growth plate, is located where the epiphysis 10 meets the metaphysic 14. The longitudinal growth of long bones occurs primarily at the epiphyseal plate 12. At the end of puberty, the epiphyseal cartilage cells stop duplicating and the entire cartilage is slowly replaced by bone, leaving only epiphyseal lines in their previous location. An epiphyseal plate 12 is located at one or both ends of the long bone between the epiphysis 10 and the diaphysis 14 (shaft) of the bone. Growth of the bone occurs towards the diaphysis 14 or shaft of the long bone. In children and young adults, an injury to the growth plate may affect the blood supply to the growth plate 12, leading to deformity of the bone and limb. Intramedullary rods locked at the proximal and distal end of a long bone, a discussed above, would cross the epiphysis 12 (growth plate), and thereby interrupt the blood supply to the epiphysis and potentially lead to bone and limb deformation. In addition, the placement of such a device would require drilling large holes through the growth plate resulting in growth disturbances or growth arrest of the growth plates.

One known solution is to insert an intramedullary rod 30 at the proximal end of the bone, but below the growth plate 12. This solution is preferred because the intramedullary rod 30 does cross the growth plate 12 and therefore does not affect the supply of blood. In reference to FIG. 3, an example of this known solution is shown. Two intramedullary rods 30 are inserted into the proximal end of the long bone, but below the growth plate 12. In this example, the two intramedullary rods 30 are inserted employing an antegrade technique below the growth plate 12 from medial and lateral entry points. The proximal ends (or leading ends) of the intramedullary rods are inserted down the axis of the medullary cavity.

In this known solution it is preferred that the intramedullary rods 30 are elastic to allow the surgeon to shape the rods 30 prior to insertion. The surgeon may shape the rods 30 to brace the fracture after the rods 30 are inserted. For example, the intramedullary rods 30 are curved so that each rod 30 bears on the inner wall of the intramedullary cavity in three points. This configuration produces flexural and translational stability and some limited axial and rotational stability. After the one or more intramedullary rods are inserted the trailing end of the intramedullary rod 30 protrudes from the bone at the insertion site 18. In most cases the trailing edge of each intramedullary rod 30 protrudes between 10 mm and 20 mm from the insertion site 18.

One known disadvantage of the above described solution is that the end of the intramedullary rod 30 protruding from the insertion site 18 is not fixed to the bone allowing the protruding end of the intramedullary rod 30 to move relative to the bone. Movement of the intramedullary rod 30 is not desirable because it can adversely affect the manner in which the fracture heals. For example, substantial movement of the protruding end of the intramedullary rod 30 may result in movement of the fracture setting, resulting in an improperly set fracture. For example, the protruding end of the of the intramedullary rod may move in the axial direction and slide back through the insertion site 18 of the bone leading to repositioning of the bone fragments in or around the fracture, and possibly leading to leg shortening.

One known solution directed at this problem is to add end caps to the protruding distal end of the intramedullary rods 30. In such systems the protruding distal end of an inserted intramedullary rod is cut so that 10 mm of rod 30 protrudes from the insertion site 18. An end cap is inserted onto the protruding rod. The cap may include threads on its exterior surface. Once the end cap is inserted onto the distal end of the intramedullary rod 30, the end cap is screwed into the insertion site of the bone 18 so that the intramedullary rod is locked into the bone. The end cap thus ensures that the intramedullary rod 30 does not slide out through the insertion site 18.

One known disadvantage of the end cap anchoring devices is that during installation lateral and rotation thrust caused by the turning of the end cap required to engage the threads with the bone at the insertion site 18 causes undesirable transverse movement of the fractured parts of the long bone due to the tendency of the intramedullary rod 30 to rotate inside the medullary cavity.

Another known disadvantage of such end cap devices is the increased expense and logistical problems caused by the requirement to store a large number of different sized devices. Patients and bones come in many different sizes and thus require different sized intramedullary rods depending on the size and length of the bone. Different sized end caps must be stored for different size rods. Likewise the distal ends of the intramedullary rods must be custom manufactured to mate with the end cap.

Another disadvantage of the end cap solution is that the installation may adversely affect the setting or placement of the intramedullary rod during installation by pushing the rod too far into the medullary cavity. As the end cap is screwed into place the end cap threads engage the bone, and the rod is exerted further into the cavity.

Another disadvantage of the known end caps is that while the end caps prevent the rod from sliding distally outward through the insertion site from the medullary cavity, they do not prevent the rod from sliding further into the medullary cavity.

Another disadvantage of known end caps is that while the end cap prevents the intramedullary rod from sliding back through the insertion site, the end cap does not prevent axial rotation of the installed intramedullary rod.

What is desired, therefore, is a device for securing the end of an intramedullary rod protruding from an insertion site in a bone so that movement of the distal end of the rod relative to the insertion site is reduced in all directions, and preferably prevented in all directions. What is further desired is a clamp for securing the distal protruding end of the rod to a bone anchor so that the distal end of the rod is fixed to the bone anchor. What is further desired is a method for installing a clamp to the distal end of an intramedullary rod wherein the rod prevents movement of the distal protruding end of the rod relative to the insertion site.

### SUMMARY OF THE INVENTION

What is desired then is a device and method that will address the aforementioned problems.

Accordingly, it is an object of the present invention to provide a device that reduces movement of an end of an intramedullary rod protruding from an insertion site in a bone.

Accordingly, it is another object of the present invention to provide a device that reduces movement of an end of an intramedullary rod protruding from an insertion site in a bone, wherein the movement is of the rod is reduced relative to the insertion site.

It is yet another object of the present invention to provide a device that reduces movement of a distal end of an intramedullary rod protruding from an insertion site in a bone, wherein the movement is of the rod is reduced relative to said device.

It is yet another object of the present invention to provide a device that reduces movement of a distal end of an intramedullary rod protruding from an insertion site in the bone wherein rotation movement of the intramedullary rod is reduced along its axis.

It is yet another object of the present invention to provide a device for reducing movement of an end of an intramedullary rod protruding from a bone, wherein a single clamping device may be used with intramedullary rods having different diameters.

Accordingly, it is an object of the present invention to provide a clamp for securing the distal end of an intramedullary rod to a bone anchor, so that distal end of the rod is fixed relative to the bone anchor.

The invention and its particular features and advantages will become more apparent from the following detailed description considered with reference to the accompanying drawings.

### BRIEF DESCRIPTION OF THE DRAWINGS

Exemplary embodiments of the invention are explained in more detail in the description which follows and are represented in the drawings, in which:

FIG. 1 shows a view of device for reducing movement of the end of an intramedullary rod protruding from an insertion site in a bone wherein the device is installed.

FIG. 2 shows a view of the device for reducing movement of an intramedullary rod protruding from an insertion site in a bone, wherein the device is installed, and wherein the portion of the intramedullary rod inside the medullary cavity of the bone is represented with broken lines.

FIG. 3 shows a prior art method for inserting an intramedullary rod into a bone, wherein the end of the rod protruding from the insertion site is not fixed relative to the bone or the insertion site.

FIG. 4A shows a clamp that comprises one component of the inventive device.

FIG. 4B shows a different view of the clamp that comprises one component of the inventive device.

FIG. 4C shows cut away view AA of the clamp, as indicated in FIG. 4B.

FIG. 5A shows a side view of a bone anchor that comprises one component of the inventive device.

FIG. 5B shows a top view of a bone anchor that comprises one component of the inventive device.

FIG. 6 shows a view of an intramedullary rod.

### DETAILED DESCRIPTION OF THE INVENTION

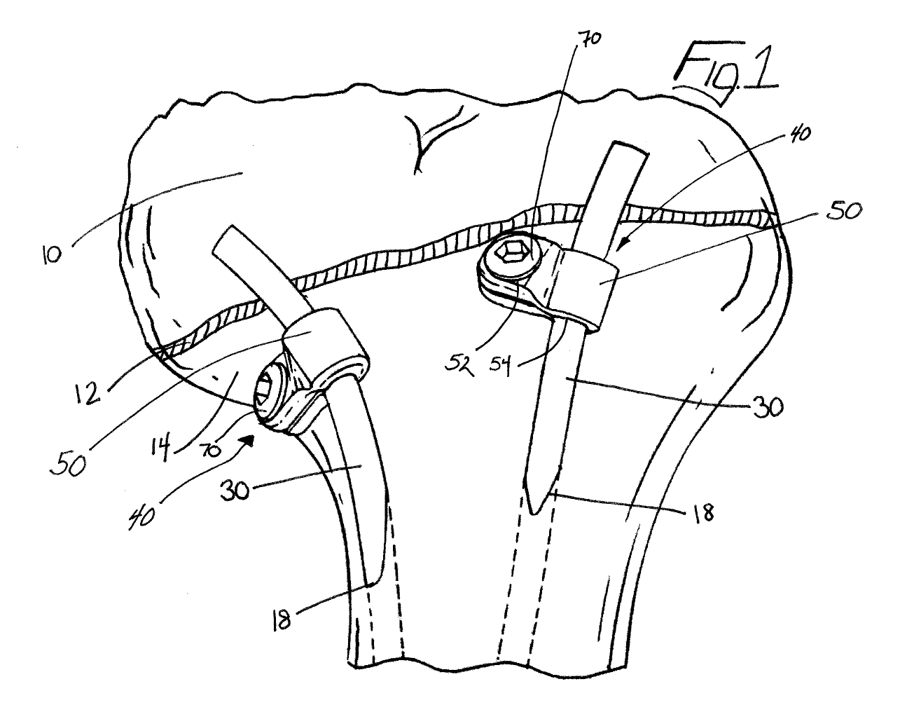

In reference to FIG. 1, one embodiment of the present invention for reducing movement of an end of an intramedullary rod 30 protruding from an insertion site 18 in a bone 10 is shown. The device 40 includes at least one clamp 50 for securing the intramedullary rod 30 protruding from the insertion site 18 in the bone 10. The securing device 40 further includes a bone anchor 70 for securing the clamp 50 to the bone 10 at a location away from the insertion site 18. Preferably the bone anchor 70 comprises a bone screw 70; however it should be understood that many different types of bone anchors may be used to secure the clamp 50 to the bone 10.

The clamp 50 comprises at least two openings 52, 54. The first opening 54 is for receiving the protruding end 32 of the intramedullary rod 30. The second opening 52 is for receiving the bone screw 70. As the bone screw 70 is inserted through the second opening 52 and into the bone 10, the bone screw 70 secures the clamp 50 relative to the bone 10. As the bone screw 70 is further inserted into the bone 10, the bone screw 70 causes the area of the first opening 54 of the clamp 50 to decrease. As the area of the first opening 54 decreases, the circumference of the first opening also decreases and fastens around the protruding end 32 of the intramedullary rod 30, thereby reducing movement of the protruding end of the intramedullary rod 30 in all directions relative to the insertion site 18.

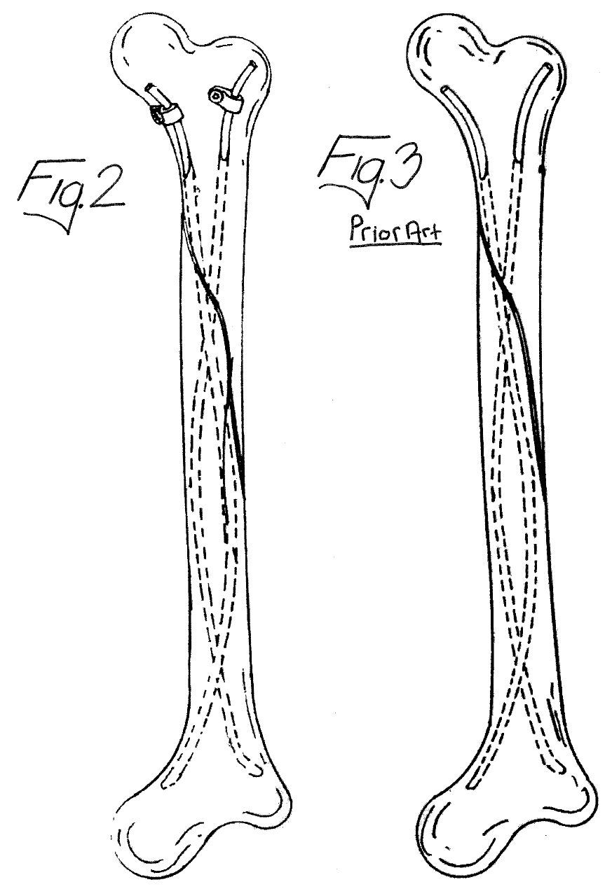

In reference to FIGS. 1-3, an embodiment of the present invention is shown in which there are two protruding intramedullary rods 30, two clamps 50 (one to secure the end 32 of each rod 30), and two accompanying bone screws 70. It should be readily apparent that many combinations are possible. For example, it may be preferred that only one intramedullary rod 30 and one corresponding securing device 40 are employed to brace a fracture in a bone 10. On the other hand it may be preferred that three or four intramedullary rods 30 and securing devices 50 are employed in a single bone 10.

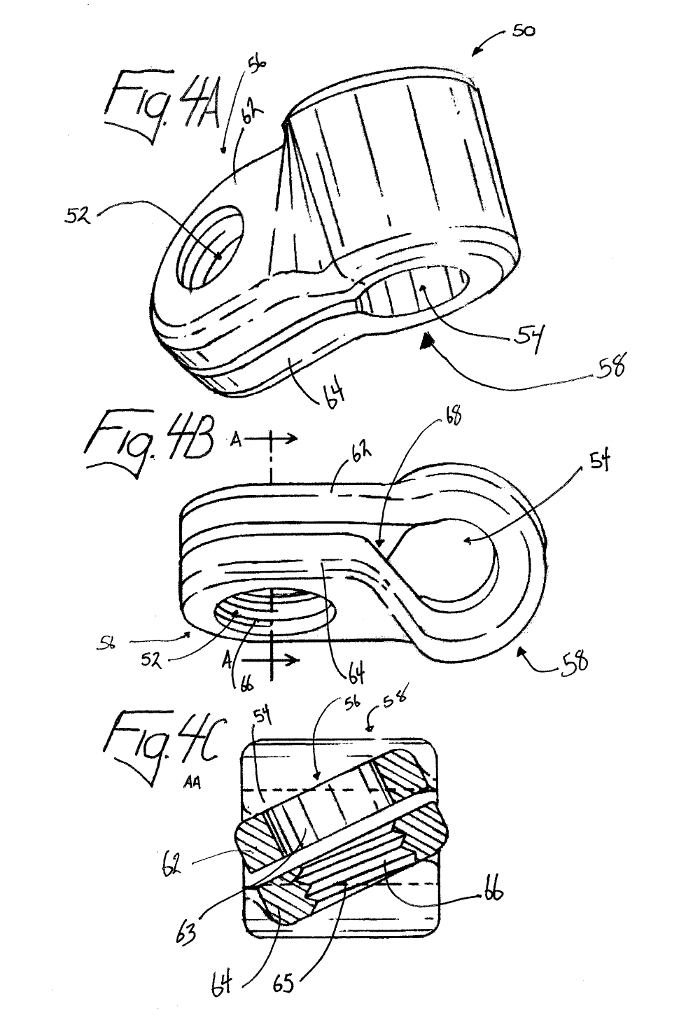

In reference to FIGS. 4A-4C, one embodiment of the clamp 50 is shown. The clamp 50 includes a first portion 58 and a second portion 56. The first portion 58 includes a first opening 54 for receiving a protruding end 32 of an intramedullary rod 30. The second portion 56 includes a second opening 52 for receiving a bone screw 70. It is preferred that the first portion 58 is on one half of the clamp 50 and the second potion 56 is on a second adjacent half of the clamp 50 as disclosed in FIGS. 4A-4C. It should be understood however, that the clamp 50 may have any configuration so long that it is operable to reduce movement of the intramedullary rod 30. The securing clamp 50 may be made from any known material for use in the body.

As shown in FIGS. 4A-4C, the clamp 50 has a first portion 58 and a second portion 56. In the embodiment shown the first portion 58 and the second portion 56 meet at a transition section 68 of the clamp 50. It is preferred that the second portion 56 of the clamp 50 comprises two, approximately parallel, spaced apart flanges 62 and 64. The parallel flanges 62, 64 protrude from the first portion 58 of the clamp 50. For example, the clamp 50 shown in FIGS. 4A-4C may be formed from a flat strip of material. The flat strip of material is rolled over its midpoint along its length so as to form a U shape. The bottom of the U comprises the first portion 58 of the clamp 50, and the top part of the U, the two spaced apart flanges 62, and 64, comprises the second portion 56 of the clamp 50.

In reference to FIGS. 4A-4C, the second portion 56 comprises a second opening 52. In the shown embodiment the second opening 52 comprises two circular openings that are coaxial when the flanges 62, 64 are parallel. FIG. 4C shows cut away view AA which shows a cut away of the second potion 56 of the clamp 50. The second opening 52 in the second portion 56 of the clamp 50 comprises two coaxial holes 63, 65. Specifically, the second opening 52 includes hole 63 in the first flange 62 and a hole 65 in the second flange 64. The bone screw 70 is inserted through the second opening 52, including hole 63 in the first flange 62, and the hole 65 in the second flange 64. After passing through the second opening 52, the screw 70 is further inserted into the bone 10. In some embodiments, as shown in FIG. 4C, the hole 65 in the second flange 64 is threaded 66 to receive corresponding threads 78 on a bone screw 70. This is one important feature of the present invention because as the bone screw 70 is inserted into the bone, threads on the bone screw 70 engage the threads 66 in the hole 65 in the second flange 64, thereby causing the second flange 64 to move toward the first flange 62. This pinching movement of the flanges 62, 64 reduces the area of the first opening 54, or in the alternative decreases the largest distance spanning the first opening 54, thereby causing the circumference of the first opening 54 to secure the previously received intramedullary rod 30.

In reference to FIGS. 4A-4C, the first opening 54 is formed between the bottom of the U of the clamp 50, and the transition area 68 between the first portion 58 and the second portion 56 of the clamp. It is preferred that the first opening 54 is formed so as to receive a distal end 32 of an intramedullary rod 30 having an approximately circular cross section. It is preferred that an axis of the first opening 54 and an axis of the second opening 52 are off set, such that the clamp 50 appears twisted at the transition area 68 between the first portion 58 and the second portion 56. In this manner the second opening 52 receives the bone screw 70 such that the bone screw 70 is approximately perpendicular to a surface of the bone 10. The axis of the first opening 54 is rotated relative the axis of the second opening 52 such that the first opening 54 may receive a distal end 32 of an intramedullary rod 30 protruding from a bone 10, wherein the protruding portion of the rod 30 is approximately parallel to the surface of the bone 10 as shown in FIG. 1, or in other embodiments offset an angle less than 90 degrees.

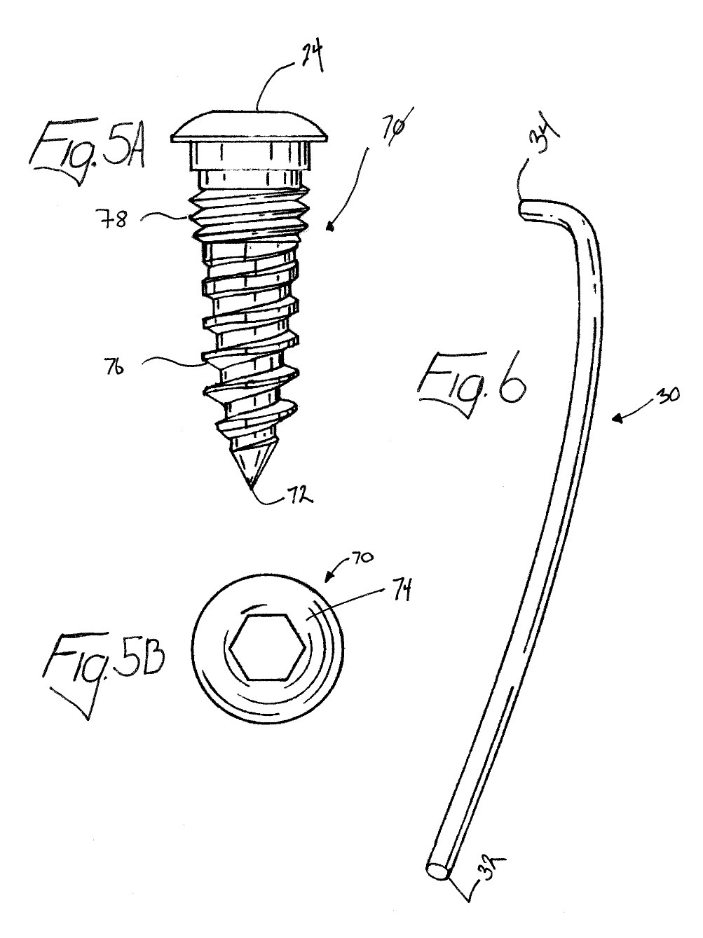

In reference to FIGS. 5A and 5B a bone screw 70 for use with the presently disclosed device 40 is shown. After installation, the bone screw 70 fixes the clamp 50 to the surface of the bone 10 at a location away from the insertion site 18. The bone screw 70 is inserted through the second opening 52 in the clamp 50 and then into the bone 10. As the bone screw 70 is inserted further into the bone 10, bone screw 70 pinches the flanges 62, 64 together, causing the decrease in the area of the first opening 54.

The bone screw 70 shown in FIGS. 5A and 5B, has a shaft along an axis. The shaft of the bone screw 70 has a distal end and a proximal end. At the proximal end of the bone screw 70 is a screw head 74. It is preferred that the screw head 74 is configured to receive a drive device, such as screw driver, to drive the bone screw 70 into bone 10. At the distal end of the bone screw 70, is a leading point 72. The leading point 72 is tapered such that distal end of the bone screw 70 comes to a signal point at the distal end of the screw 70. The leading point 72 allows the surgeon to more easily initiate entry of the bone screw 70 into a bone 10.

The bone screw 70 shown in FIGS. 5A and 5B has two sets of threads 76, 78 on the shaft of the bone screw 70. The bone screw 70 has a first set of threads 78 on the shaft proximate to the screw head 74. The bone screw 70 has a second set of threads 76 on the shaft proximate to the leading point 72 of the bone screw 70. It is preferred that the second set of threads 76 are sized to engage with bone 10. After the bone screw 70 is inserted into the bone 10, the second set of threads 76 prevent movement of the bone screw 70 relative to the bone 10, thereby maintaining the position of the clamp relative to the insertion site 18 of the intramedullary rod 30.

The first set of threads 78 are preferably sized to engage with the clamp 50. Specifically, the first set of threads 78 are sized to engage with the receiving threads 66 in the second hole 65 in the lower flange 64 of the clamp 50. It should be noted that in this configuration the second set of threads 76 on the bone screw 70 should be sized to pass through the receiving threads 66 in the second hole 65 in the lower flange 64 of the clamp 50. The bone screw 70 may be constructed from a metal material that is approved for use in humans, however the bone screw 70 may be constructed from any suitable material for use in bone.

In reference to FIG. 6, an intramedullary rod 30 for use with the present invention is shown. Different types of intramedullary rods 30 and their uses are well known. Typically an intramedullary rod 30 has a long slender shaft. The rod 30 further includes a distal end 32 and a proximal end 34 at either end of the rod 30. Typically the intramedullary rod 30 has a circular cross section. Intramedullary rods 30 are generally constructed from a suitable metal material, such as titanium; however rods 30 may be constructed from any suitable material for use in humans. It is preferred that in some embodiments of the present invention the intramedullary rods 30 are elastic or flexible so that the surgeon can shape the intramedullary rod 30 prior to inserting the intramedullary rod 30 into the medullary canal.

In FIGS. 1 and 2, the clamping device 40 is shown installed to the surface if the bone 10. In FIG. 2, two intramedullary rods are shown inserted into the medullary cavity of a long bone. FIG. 2 shows the inserted portions of the intramedullary rod 30 with broken lines. FIGS. 1 and 2 show an embodiment in which two-intramedullary rods are used, however the number of intramedullary rods used to secure a fracture may vary.

To install an intramedullary rod employing the disclosed clamping device 40 a surgeon first drills hole into the bone 40 to form the insertion site 18. The hole forms a passage between the exterior of the bone and the interior of the medullary cavity. Next, the surgeon inserts the intramedullary rod 30 into and along the intramedullary cavity via the insertion site 18. After the intramedullary rod 30 is fully installed a portion of the rod 32 protrudes from the insertion site. The surgeon uses the clamping device 40 to reduce movement of the protruding end 32 of the intramedullary rod 30 in all directions.

After the rod 30 is installed, the surgeon takes a clamp 50 and inserts it onto the protruding end 32 of the intramedullary rod 30. The surgeon inserts the protruding end 32 of the rod through the first opening 54 in the first portion 58 of the clamp 50. After the clamp 50 is installed on to the rod 30, the surgeon uses a bone screw 70 to fix the clamp 50 to the bone 10. The surgeon should locate the clamp 50 at a position above the insertion site 18, but below the growth plate 12 of the bone 10 as shown in FIG. 1. Next the surgeon inserts the leading point 72 of the bone screw 70 through the first opening 52 in the second potion 56 of the clamp 50. After the leading point 72 is inserted into the through the second opening 52, the surgeon drives the bone screw 70 into the bone 10. As the surgeon continues to drill the bone screw 70 into the bone 10, the bone screw fixes the clamp to the surface of the bone.

As the bone screw 70 is driven into the bone 10, the firsts set of bone screw threads 78 engage with threads 66 in the hole 65 of the lower flange 64 in the second portion of the clamp 50. As the bone screw 70 is further driven into the bone 10, the engaged threads cause the lower flange 64 to move upward toward said upper flange 62 and toward the screw head 74. The downward force of the screw head 74 causes the upper flange 62 to move toward the lower flange 64 at the same time. As the flanges 62, 64 of the clamp 50 are drawn together the area of the first opening 54 in the first portion 58 of the clamp 50 is reduced. The reduction in area of this first opening 54 causes the first opening 54, or the circumference thereof, to securely grip the protruding end 32 of the intramedullary rod 30 protruding from the insertion point 18. The surgeon keeps driving the bone screw 70 into the bone, until the clamp 50 has securely gripped the intramedullary rod 30, and the clamp 50 is secured to the bone 10. After installed, the clamp 50 reduces movement of the protruding end 32 of the intramedullary rod 30 in all directions relative to the position of the insertion site. It is preferred that the clamp prevents movement of the protruding end of the intramedullary rod 30 in all directions, however the clamp still achieves beneficial effects if it reduces movement of the rod in all directions. After installation, the surgeon may clip the protruding end 32 of the intramedullary rod that extends beyond the installed clamp 50.

It should be understood by a person of ordinary skill in the art that the located of the threads on the clamp may be varied, or that no threads are needed. For example, in some embodiments neither hole 63, 65 has threads, nor does the bone screw have a first set of threads. As the bone screw is driven into the bone the force of the bone and counteracting force of the screw head 74 causes the flanges to pinch together and thereby reduce the area of the first opening 54.

Although the invention has been described with reference to several embodiments with certain constructions, structures, ingredients and formulations and the like, these are not intended to exhaust all possible arrangements or features, and indeed many other modifications and variations will be ascertainable to those of skill in the art.

What is claimed is:

1. A device for reducing movement of an intramedullary rod protruding from an insertion site in a bone, said device comprising:
a bone anchor,
a clamp having a first portion and a second portion, said first portion comprising a first opening for receiving a protruding end of said intramedullary rod, and said second portion comprising a second opening for receiving said bone anchor; and
wherein said device reduces movement of said protruding end of said intramedullary rod relative to said intramedullary rod insertion site when said protruding end is received by said first opening, said bone anchor is received by said second opening, and said bone anchor is inserted into said bone.

2. The device of claim 1, wherein said bone anchor has a longitudinal axis, a proximal end, a distal end, and a first set of threads, and
wherein said first set of threads engage said second opening when said bone anchor is received in said second opening and inserted into said bone.

3. The device of claim 2, wherein said second portion comprises first and second spaced apart flanges moveable between angled and parallel orientations relative to one another.

4. The device of claim 3, wherein said second opening comprises a first hole in said first flange, and a second hole in said second flange.

5. The device of claim 4, wherein one or more of said first hole and said second hole comprises receiving threads configured to receive said first set of threads of said anchor.

6. The device of claim 5, wherein said anchor comprises a second set of threads configured to engage said bone.

7. The device of claim 6, wherein said first set of threads are proximate to said proximal end of said bone anchor, and said second set of threads are proximate to said distal end of said bone anchor.

8. The device of claim 7, wherein said second set of threads are configured to not engage said receiving threads when said second set of threads of said bone anchor are inserted through said second opening.

9. An intramedullary rod system, said system comprising:
an intramedullary rod;
a bone anchor extending along a longitudinal axis, said anchor having a proximal end and a distal end, said anchor comprising a first set of threads;
a clamp having a first portion and a second portion, said first portion comprising a first opening for receiving a protruding end of said intramedullary rod, and said second portion comprising a second opening for receiving said bone anchor; and
wherein said system reduces movement of said protruding end of said intramedullary rod relative to said an insertion site of said intramedullary rod when said protruding end is received by said first opening, said bone anchor is received by said second opening, and said bone anchor is inserted into said bone.

10. The intramedullary rod system of claim 9, wherein said second portion comprises first and second spaced apart flanges moveable between angled and parallel orientations relative to one another.

11. The intramedullary rod system of claim 10, wherein said second opening comprises a first hole in said first flange, and a second hole in said second flange.

12. The intramedullary rod system of claim 11, wherein one or more of said first hole and said second hole comprise receiving threads configured to receive said first set of threads of said anchor.

13. The intramedullary rod system of claim 12, wherein said bone anchor comprises a second set of threads configured to engage bone, and
wherein said first set of threads are proximate to said proximal end of said bone anchor, and said second set of threads are proximate to said distal end of said bone anchor.

14. The intramedullary rod system of claim 13, wherein said second set of threads are configured to not engage said receiving threads when said second set of threads of said bone anchor are inserted through said second opening.

15. A method for reducing movement of an end of an intramedullary rod protruding from an insertion site in a bone, the method comprising the steps of:
providing an intramedullary rod;
providing a bone anchor;
providing a clamp having a first opening for receiving said intramedullary rod, and a second opening for receiving said bone anchor;
inserting said intramedullary rod into an insertion site in a bone so that a portion of said intramedullary protrudes from said bone;
inserting said protruding portion of said intramedullary rod through said first opening of said clamp;
inserting said bone anchor through said second opening of said clamp; and;
inserting said bone anchor into said bone; and
wherein movement of said protruding end of said intramedullary rod is reduced relative to said bone anchor after said protruding end is received by said first opening, said bone anchor is received by said second opening, and said bone anchor is inserted into said bone.

16. The method of claim 15, further comprising the steps of:
providing a first set of threads on said bone anchor.

17. The method of claim 16 further comprising the step of:
providing receiving threads in said second opening configured to receive said first threads on said bone anchor; and
wherein when said bone anchor is inserted through said second opening of said clamp, said receiving threads receive said first set of threads.

18. The method of claim 17 further comprising the step of;
providing a second set of threads on said bone anchor, set second set of threads configured to engage the bone.

19. The method of claim 18, further comprising the steps of:
screwing said second set of threads of said bone into said bone.

20. The method of claim 15, wherein when said clamp prevents movement of said protruding end of said intramedullary rod relative to said bone anchor after installation.
